(19) United States
(12) Patent Application Publication
(10) Pub. No.: US 2016/0369221 A1
(43) Pub. Date: Dec. 22, 2016

(54) FLUIDIC DEVICE AND PERFUSION SYSTEM FOR IN VITRO COMPLEX LIVING TISSUE RECONSTRUCTION

(71) Applicant: Mikhail Alexandrovich PONOMARENKO, 's-Hertogenbosch (NL)

(72) Inventor: Mikhail Alexandrovich PONOMARENKO, 's-Hertogenbosch (NL)

(21) Appl. No.: 15/101,861

(22) PCT Filed: Dec. 2, 2014
(86) PCT No.: PCT/NL2014/050824

§ 371 Date: Jun. 3, 2016

(30) Foreign Application Priority Data
Dec. 4, 2013 (NL) 2011895

(51) Int. Cl. C12M 1/00; C12N 5/071; C12M 1/12; C12M 3/00

(52) U.S. Cl. CPC C12M 29/10; C12M 21/08; C12N 5/0697; C12M 23/34; C12M 23/06; C12M 25/12; C12M 47/02

(57) ABSTRACT

The present invention relates to a fluidic device for in vitro complex living tissue reconstruction comprising at least one set of distinct compartments, which set comprises at least a first, second and a third compartment and a separating material separating the compartments comprised in the set of distinct compartments from one another, wherein the at least one set of distinct compartments defines at least one exchange region in which the compartments comprised in the set congregate and wherein at least a part of the separating comprised in the at least one exchange region is configured such that direct communication is allowed between each of the compartments comprised in the at least one set of distinct compartments with one another. The present invention also relates to the use of the fluidic device of the present invention. The present invention further relates to a perfusion system comprising the fluidic device and to a method for in vitro culturing and/or co-culturing, including complex living tissue reconstruction using the fluidic device and/or perfusion system of the present invention, as well as to a hollow membrane and use of the hollow membrane for culturing, co-culturing, evaluating, sampling and/or harvesting of cells, circulatory system cells, neuronal cells and/or interstitial cells, products and/or metabolites from the fluidic device of the present invention.

The present invention relates to a fluidic device and a perfusion system for in vitro complex living tissue reconstruction. The present invention also relates to the use of the fluidic device of the present invention for culturing and/or co-culturing, evaluating, sampling and/or harvesting of tissue cells, circulatory system cells and neuronal cells, interstitial cells, products and/or metabolites from the fluidic device. The present invention relates to the use of the fluidic device of the present invention for culturing and/or co-culturing, evaluating, sampling and/or harvesting of non-cellular, unicellular and/or multicellular organism and/or tissue, material, products and/or metabolites from the fluidic device other than the reconstructed tissue. The present invention further relates to a method for in vitro culturing and/or co-culturing cells, including complex living tissue reconstruction using the fluidic device of the present invention, as well as to a hollow membrane and use of the hollow membrane for culturing, co-culturing, evaluating, sampling and/or harvesting of tissue cells, circulatory system cells, neuronal cells, interstitial cells and/or products and/or metabolites from the fluidic device of the present invention.

Advances in medical genetics and human genetics have enabled a more detailed understanding of the impact of genetics in disease. Large collaborative research projects (e.g. the Human genome project) have laid the groundwork for the understanding of the roles of genes in normal human development and physiology, revealed single nucleotide polymorphisms (SNPs) that account for some of the genetic variability between individuals, and made possible the use of genome-wide association studies (GWAS) to examine genetic variation and risk for many common diseases.

The use of genetic information has played a major role in developing personalized medicine, i.e. the customization of healthcare—with medical decisions, practices, and/or products being tailored to the individual patient. Examples of personalized medicine can be found in, for example, the field of oncology, wherein personalized cancer management include the testing for disease-causing mutations in the breast cancer type 1 (BRCA1) and breast cancer type 2 (BRCA2) genes, which are implicated in hereditary breast-ovarian cancer syndromes.

Furthermore, personalized medicine can also be found in the field of organ transplantation. Transplantation medicine is one of the most challenging and complex areas of modern medicine. Some of the key areas for medical management are the problems of transplant rejection, during which the body has an immune response to the transplanted organ, possibly leading to transplant failure and the need to immediately remove the organ from the recipient. When possible, transplant rejection can be reduced through serotyping to determine the most appropriate donor-recipient match and through the use of immunosuppressant drugs. The emerging field of regenerative medicine is allowing scientists and bioengineers to create organs to be re-grown from the patient's own cells (stem cells, or cells extracted from the failing organs).

Regenerative medicine also empowers scientists to grow tissues and organs in the laboratory and safely implant them when the body cannot heal itself. Importantly, regenerative medicine has the potential to solve the problem of the shortage of organs available for donation compared to the number of patients that require life-saving organ transplantation. Depending on the source of cells, it can potentially solve the problem of organ transplant rejection if the organ's cells are derived from the patient's own tissue or cells. However, the current application of regenerative medicine is limited and the (re)construction of organs is still labour-intensive.

Also in drug development, e.g. drug discovery, the role of personalized medicine is of increasing importance. Drug development has been hampered because it relies on the use of animal models that are costly, labour-intensive, time-consuming and questionable ethically. Of even greater concern is that animal models often do not predict results obtained in humans, and this is a particular problem when addressing challenges relating to metabolism, transport and oral absorption of drugs and nutrients.

The present invention provides a fluidic device for in vitro complex living tissue reconstruction. It is proposed that the in vitro complex living tissue reconstructed by the fluidic device of the present invention closely mimics the in vivo tissue of a living multicellular organism. The present invention provides hereto a fluidic device for in vitro complex living tissue reconstruction comprising:
at least one set of distinct compartments, such as a set of channels and/or microchannels, which set comprises at least channel first, a second and a third compartment; and
a separating material separating the compartments comprised in the set of distinct compartments from one another, wherein:
the at least one set of distinct compartments defines at least one exchange region in which the compartment comprised in the set congregate; and
at least a part of the separating material comprised in the at least one exchange region is configured such that direct communication is allowed between each of the compartments comprised in the at least one set of distinct compartments with one another.

The fluidic device of the present invention provides a simple and elegant cell coculture in vitro model wherein the in vitro reconstruction of human, animal and/or plant tissue, e.g. complex living tissue, closely resembles the construction of human and/or animal tissue in vivo, e.g. in structure (morphology) and in function. The fluidic device of the present invention provides a system wherein cellular communication between the different cell cultures (including immune cells) is allowed via direct contact, i.e. juxtacrine signalling, communication over a short distance, i.e. paracrine signalling, and/or communication over a relatively longer distance, i.e. endocrine signalling by mimicking the juxtacrine, paracrine and/or endocrine signalling in the fluidic device, the present invention provides a cell coculture model which mimics the complex in vivo like structure and function of a tissue of a living multicellular organism. The fluidic device of the present invention allows scientists/bioengineers to evaluate the formed structure of the in vitro reconstructed tissue, e.g. via coculturing, sampling, harvesting or the like, and to evaluate the function of the in vitro reconstructed tissue, e.g. via genomics, transcriptomics, proteomics, metabolomics or the like. The fluidic device of the present invention further allows culturing, co-culturing, evaluating, sampling and/or harvesting non-cellular, unicellular, multicellular organisms and/or tissue, material, products and/or metabolites, e.g. intestinal microbiota, biomedical materials or the like, from the fluidic device other than the reconstructed tissue. The human and/or animal models known in the art do not provide an in vitro model wherein both the structure and function of a reconstructed tissue as well as responses to coculture with a guest organism and/or material can be studied. In fact, none of the in vitro models known in the art provide a fluidic device wherein the reconstruction of human and/or animal tissue is regulated, coordinated and integrated by providing a neurohumoral regulation. However, the fluidic device of the present invention, comprising at least one set of distinct compartments, which set comprises at least a first, a second and a third compartment, provides an in vitro reconstruction of a human and/or animal tissue wherein the (to be) reconstructed tissue is regulated by a neurohumoral regulation.

As used herein the “fluidic device” refers to a device of any size or orientation which comprises one or more sets of distinct compartments and is suitable for the culture of living cells. A fluidic device can be capable of moving any amount of fluid within the fluid flow ranges described herein below, e.g. a fluidic device can be a microfluidic device or a device capable of moving larger volumes of fluid.

Furthermore, as used herein the term “direct communication” refers to the possibility to exchange cells, compounds, products and/or metabolites between each of the distinct compartments. Also, the term “direct communication” refers to possibility of, for example, neuronal cells to extend outside channel compartment comprising the neuronal cells by formation of neurites, e.g. an axon and/or a dendrite.

As used herein, the term “compartment” refers to any capillary, channel, tube, or groove that is deposed within or upon a substrate. A compartment can be a microchannel; i.e. a channel that is sized for passing through microvolumes of liquid. The compartments of the fluidic device of the present invention may have any suitable form. In an embodiment of the present invention, the fluidic device comprises at least one set of distinct compartments, wherein the compartments are substantially tubular, to form a tubular fluidic device, or substantially rectangular, to form a planar fluidic device. It is noted that the compartments of the present invention may be a triangular prism, a pentagonal prism, a hexagonal prism, and the like. It is further noted that a combination of different forms may be used. In an embodiment of the present invention, the fluidic device comprises at least one set of distinct compartments having a substantially tubular form which set of distinct compartments is combined with compartments with a substantially triangular prism form.

It is now proposed that by providing a fluidic device according to the present invention wherein the fluidic device comprises at least one set of distinct compartments, which set comprises at least three compartments and wherein the three compartments are in communication with one another, the in vitro reconstruction of (complex) living tissue, e.g. human and/or animal tissue, closely resembles the way living tissue occurs in vivo, i.e. in nature. By mimicking the in vivo method of reconstruction of living tissue, the in vitro reconstructed living tissue mimics the in vivo living tissue more closely and more precisely than compared to in vitro methods of reconstruction of living tissue known so far. The essence of the present invention resides in the reconstruction of living tissue by the distinct characteristics of the three distinct compartments and the possibility to communicate with one another through the separating material separating the at least three compartments. To allow the tissue cells, circulatory system cells and, optionally, neuronal cells to communicate with one another, it is possible to create an in vitro cell coculture, which closely resembles the natural environment of the living tissue to be reconstructed.

Even further, by providing different distinct compartments having distinct functionality, it is possible to reconstruct tissue in vitro based on tissue cells, circulatory system cells and, optionally, neuronal cells extracted from the same unique multicellular living organism, e.g. human being. The fluidic device of the present invention therefore provides a method for the reconstruction of unique living tissue each time the fluidic device of the present invention is seeded with cell culture material. As a consequence, the tissue constructed by the fluidic device of the present invention may closely resemble the natural tissue of a multicellular living organism and provide therefore an in vitro alternative method which empowers scientists/bioengineers to grow different types of tissue and/or organs, e.g. skin, stomach, intestine, muscles, bone, adipose tissue or the like, as well as to support culture other than reconstructed tissue non-cellular, unicellular, multicellular organism, tissue and/or materials for scientific and industrial needs. It should be noted that the fluidic device of the present invention may construct any kind of living tissue, e.g. mammal tissue such as human and/or animal tissue.

Additionally, the fluidic device of the present invention empowers scientists/bioengineers to construct patient-unique tissue in order to select the most promising treatment therapy for a specific individual. It has to be understood that the fluidic device of the present invention further provides also a method to construct complex living tissues which can be used in the drug development to select the most promising drug candidates. Thus, the use of the fluidic device of the present invention for in vitro reconstruction of human tissues may reduce and/or replace the application of animal models in drug discovery. The coculture of tissue cells, circulatory system cells and, optionally, neuronal cells provided by the fluidic device of the present invention empowers the scientists/bioengineers to design desired types of in-vivo-like living tissue in vitro and therefore offers a more promising test-model of a desired multicellular organism drug compared to in vivo and/or in vitro models used nowadays.

The tissue cells may comprise a wide variety of living tissue cells, e.g. human and/or animal tissue cells. The tissue cells may be selected from the group consisting of primary cells, cells, cultured cells, passaged cells, immortalized cells, transgenic cells, genetically modified cells, cancerous cells or cells from a multicellular organism with a cancer, cells from a multicellular organism with disease or disorder, stem cells, embryonic stem cells (ESCs), induced pluripotent stem cells (IPSCs), tissue-specific progenitor/stem cells. The tissue cells may be selected from the cells derived from tissue and/or organoid, i.e. a structure that resembles an organ, of a desired multicellular organism.

The circulatory system cells, such as blood, vascular and/or lymphatic system cells, may be selected from the group consisting of primary blood and/or (lymph)endothelial cells, primary pericytes, cells, cultured cells, passaged cells, immortalized cells, transgenic cells, genetically modified cells, cancerous cells or cells from a multicellular organism with a cancer, cells from a multicellular organism and/or organoids with disease or disorder, stem cells, ESCs, IPSCs, tissue-specific progenitor/stem cells, peripheral blood mononuclear cells (PBMC), plasmacytoid dendritic cells (PDC), myeloid dendritic cells (MDC), B cells, macrophages, monocytes, natural killer cells, NKT cells, CD4+ T cells, CD8+ T cells, granulocytes or precursors thereof. The circulatory system cells may be derived from a desired multicellular organism.

The neuronal cells may be selected from the group consisting of primary cells, cells, cultured cells, passaged cells, immortalized cells, transgenic cells, genetically modified cells, cancerous cells or cells from a multicellular organism with a cancer, cells from a multicellular organism and/or organoids with disease or disorder, stem cells, ESCs, IPSCs, tissue-specific progenitor/stem cells, unipolar or pseudounipolar cells, bipolar cells and/or multipolar cells (e.g. Golgi I and Golgi II). The neuronal cells may further be selected from the group consisiting of basket cells, betz cells, lugaro cells, medium spiny neurons, purkinje cells, pyramidal cells, renshaw cells, unipolar brush cells, granule cells, anterior horn cells or spindle cells. The neural cells may also be derived from a desired multicellular organism.

The separating material may be made of an impermeable, permeable and/or semi-permeable material. In case the separating material is made of an impermeable material, the separating material may comprises at least one area having a plurality of pores and/or passages. It is noted that the area having a plurality of pores is at least located at the exchange region as defined above. By providing a fluidic device wherein the at least three compartments are separated by a separating material made of a material comprising at least one area having a plurality of pores, the three compartments are able to communicate with one another at the at least one exchange region where the at least three compartments congregate. The size of the pores may be chosen such that the communication is in one-way direction or in a two-way direction. The pattern of the pores between the different compartments may be chosen such that different areas of the material where the separating material may be made of provide different functionality with regard to the permeability of the material. It is even possible to define the size of the pores in such way that the pores connecting the first compartment and the second compartment are different compared to the size of the pores connecting the second compartment and the third compartment and even further different compared to the size of the pores connecting the third compartment and the first compartment.

The pore aperture in the material where the separating material may be made of separating the at least three compartments from one another in the above defined at least one exchange region depends on the specific needs of the tissue to be reconstructed. Preferably the pores of the area comprised by the separating material may be between about 0.5 μm and about 10 μm in diameter. Preferably, the pores of the material may be about 8 μm or about 1 μm in diameter. In case transmigration of cells across the material (e.g. chemotaxis and/or motility studies), is desired, pores of about 5 μm in diameter are particularly useful. As already described above, the pores of the material can be varied per area of the material. Furthermore, the pores of the material can be irregularly and/or regularly spaced. Even the distance between the pores can vary. Preferable the pores in the material may be 0.1 μm or further apart, more preferably 1 μm apart, 5 μm apart, 10 μm apart, 15 μm apart, 20 μm apart, 25 μm apart, 50 μm apart, 100 μm apart, 1000 μm apart or even further apart.

The area having a plurality of pores may be made of a permeable and/or semi-permeable material, e.g. a membrane, micro-carrier beads, self-assembling micro- and nanofluidic devices or a matrix. As already explained above, the permeability of the permeable and/or semi-permeable material may be varied between the different compartments. Also, the permeability of the permeable and/or semi-permeable material may be varied per area of the permeable and/or semi-permeable material itself. The separating material may also be formed by a permeable and/or semi-permeable material, e.g. the material as described above entirely consist of a permeable and/or semi-permeable material. Again, it should be noted that the permeability of the permeable and/or semi-permeable material and the pattern of the permeability of the permeable and/or semi-permeable material may be varied between the different compartments.

The above defined permeable and/or semi-permeable material separating the at least three compartments from one another, at least in the exchange region in which the compartments comprised in the set congregate, may have the form of a permeable and/or semi-permeable matrix. Preferably the permeable and/or semi-permeable matrix may be located in such way that the matrix is in direct connection with the at least three compartments. The use of such a matrix is particularly applicable in a fluidic device having a planar channel structure wherein the fluidic device is divided into at least three different compartments wherein the separating material comprising the matrix separating the at least three compartments having a T, X, H, U- or Y-shaped form. The matrix may be preferably located at least at the exchange region, i.e. the junction area, of the separating material allowing the at least three compartments to communicate with one another.

In a preferred embodiment of the present invention, the separating material is configured such that it encloses an interstitial space. The interstitial space is preferably located between the at least three compartments and may have the form of a fluid channel configured to comprise a fluid or a gel. It is noted that at least a part of the separating material, e.g. the fluid channel, comprised in the at least one exchange region comprises a plurality of passages configured to allow mass transfer, such as cell migration, between each of the compartments comprised in the at least one set of distinct compartments with one another. The plurality of passages may have the form of pores provided in the fluid channel, or may have the form of pillars in case the compartments and separating material, e.g. in the form of a channel, are embedded in a suitable material the fluidic device is formed of.

The separating material may be at least partially made of a biodegradable or non-biodegradable material. In other words, the material of the separating material comprising at least one area having a plurality of pores may be biodegradable or non-biodegradable. The biodegradability of the material can be varied between the different compartments. By providing a fluidic device comprising at least three distinct compartments wherein the at least three compartments are separated by a biodegradable material, the present invention therefore provides the possibility to design complex structures of biodegradable material in order to reconstruct complex living tissue, e.g. mammal organs. By providing a fluidic device wherein the material separating the at least three compartments is made of a biodegradable material, the resulting reconstructed tissue may have a three-dimensional structure wherein separating material and/or the area having a plurality of pores (e.g. membrane and/or matrix), is no longer present.

The at least three compartment structure of the fluidic device of the present invention may be designed by using an intelligent design unit, e.g. a computer, using a 3D printer to actual print the three-dimensional fluidic device comprising the at least three compartments separated from one another by a separating material, e.g. a material comprising at least one area having a plurality of pores. However, other methods such as etching, machining or micro-machining may be suitable as well. After seeding the tissue cells, circulatory system cells and, optionally, neuronal cells to the corresponding compartments and/or channels, the tissue can be reconstructed in a three-dimensional way. Such three-dimensional reconstruction of, for example, a complex living tissue empowers the scientist/bioengineer to reconstruct in vitro a patient specific complex tissue, e.g. an organ, such as skin or intestine reconstructed with patient specific tissue which may be used for organ transplantation.

Even further, the fluidic device of the present invention may be formed by a solid material comprising at least partially a semi-permeable and/or permeable material wherein at least one set of distinct compartments is created, e.g. by providing boreholes into the solid material comprising at least partially a semi-permeable and/or permeable material.

In an embodiment of the present invention, the fluidic device comprises at least one set of distinct compartments wherein each of the at least three compartments comprised in the set define an inner surface enclosing the interior of the compartment and an outer surface adjacent to the inner surface of the compartment facing at least a part of the outer surface of the at least two other compartments. In such embodiment, the at least three compartments may be formed by using a material, e.g. the above described permeable and/or semi-permeable membrane, enclosing the respective compartment which compartment is physically separated from the at least two other compartments. As a consequence, the materials enclosing the at least three physically separated compartments may be different from one another. At least a part of the outer surfaces of the physically separated compartments may be located at a minimal distance from one another. Preferably, the minimal distance between each of the compartments, e.g. the outer surface of the compartments, in the at least one exchange region does not exceed 1000 μm. In an even further preferred embodiment of the present invention, the minimal distance does not exceed 500 μm, preferably not exceed 400 μm, preferably not exceed 300 μm or preferably lies within the range of 10 to 250 μm.

In a favourable embodiment of the present invention the minimal distance between the outer surfaces of the physically separated compartments does not exceed 1000 μm, since by a minimal distance between the outer surfaces of greater than 1000 μm direct contact communication between the separated compartments (e.g. juxtacrine signalling) is hindered. Preferably, the minimal distance between the outer surfaces of the physically separated compartments may be in the range from 0 μm to about 500 μm. More preferably, the minimal distance between the outer surfaces of the physically separated compartments may be in the range from about 5 μm to about 10 μm. In a further favourable embodiment of the present invention, at least a part of the outer surface of a physically separated compartments may comprise a surface which contacts with at least a part of the outer surfaces of the at least other two compartments, i.e. a minimal distance between the outer surfaces of the physically separated compartments of 0 μm.

As already indicated above, the separating material of the fluidic device may enclose an interstitial space. However, in a further embodiment of the present invention, the fluidic device may comprise at least one interstitial space enclosed by the outer surfaces of the at least three compartments. The interstitial space may also be formed naturally between the outer surfaces of the at least three compartments. In an even further embodiment, the at least one interstitial space is being arranged for receiving interstitial cells, products and/or metabolites, e.g. signalling molecules comprised in the interstitial fluid, forming an interstitial space.

The interstitial space may comprise an extracellular matrix (ECM), e.g. basement membranes and/or interstitial fluid produced by cells to be comprised into the first, second and/or third compartment, and/or the interstitial cells to be comprised on and/or into the separating material. In an embodiment of the present invention, the interstitial cells may be selected from the group consisting of resident and wandering primary cells of connective tissue, cells, cultured cells, passaged cells, immortalized cells, transgenic cells, genetically modified cells, cancerous cells or cells from a multicellular organism with a cancer, cells from a multicellular organism and/or organoids with disease or disorder, stem cells, ESCs, IPSCs, tissue-specific progenitor/stem cells, fibroblasts, fibrocytes, reticular cells, tendon cells, myofibroblasts, adipocytes, melanocytes, mast cells, macrophages. The cells of the connective tissue may be derived from a desired multicellular organism.

The products and/or metabolites may further comprise a water solvent comprising sugars, salts, fatty acids, amino acids, coenzymes, signalling molecules, hormones, neurotransmitters, mucus, unicellular, multicellular and/or non-cellular organisms, e.g. intestinal microbiota, as well as waste products and/or cellular metabolites from human, animal and/or guest organism, e.g. intestinal commensal and/or pathogen microbiota.

The interstitial fluid may further comprise blood plasma without the plasma proteins and may also comprise some types of wandering cells, e.g. white blood cells.

In even a further embodiment of the present invention, the at least one interstitial space comprises at least one fluid channel wherein the fluid channel is in communication with the at least one set of distinct compartments.

The interstitial space may be formed entirely of a plurality of fluid channels wherein each of the fluid channels is in communication with at least one set of distinct compartments. Favourably, the fluid channels may be arranged to receive interstitial cells, products and/or metabolites, e.g. the interstitial fluid. By providing an interstitial space comprising at least one fluid channel, the present invention empowers scientists/bioengineers to design more complex fluidic devices wherein the location and therefore the accessibility of interstitial cells, products and/or metabolites is controllable. In a further embodiment of the present invention, the fluid channel is made of a permeable and/or semi-permeable material, e.g. membrane. The fluid channel of the present invention may be made of a biodegradable or non-biodegradable material. The pore aperture, the porosity and/or molecular weight cut off (MWCO) of the material of the interstitial fluid channel depend on the size of the compounds desirable to separate from the interstitial space. By defining the permeability of the fluid channel, wherein the fluid channel optionally comprises products and/or metabolites, e.g. interstitial fluid, the access of tissue cells, circulatory system cells and, optionally, neuronal cells can be controlled.

In an embodiment of the present invention, the at least one set of distinct compartments may further comprise a fourth compartment.

In a further embodiment of the present invention, the fluidic device may comprise two or more sets of distinct compartments wherein each of the sets comprising at least three compartments. It is noted that in each set the separated compartments are in direct communication with one another in the at least one exchange region and, optionally, the two or more sets of distinct compartments are in direct communication with one another. Since the fluidic device of the present invention is not restricted to one particular set of at least three compartments, the reconstruction of complex living tissues, e.g. organs, is one of the possibilities provided by the fluidic device of the present invention. It is even possible to reconstruct patient specific healthy body tissue and patient specific body tissue affected with a certain disease in one single fluidic device. Such fluidic device may be useful in selecting the most optimal patient unique therapy wherein the affected tissue is cured and wherein the healthy body tissue of the patient is unaffected by the chosen treatment.

In an even further embodiment of the present invention, the fluidic device at least comprises a first set of distinct compartments and a second set of distinct compartments wherein at least one, but preferably two, of the compartments of the first set are also part of the second set. By allowing one or more compartments to be part of a first and second set of distinct compartments, direct communication between the sets is more likely.

The fluidic device of the present invention as well as the at least one set of distinct compartments may have any particular form, preferably a planar and/or tubular form. The fluidic device may be any pressure resistant capillary, channel, tube, groove, chamber, container, reservoir or the like. It is noted that a planar shaped fluidic device is preferred to perform a dynamical (i.e. live) visual control of the coculture, e.g. by fluorescent microscopy, to evaluate tissue integrity and/or permeability, e.g. by measuring trans-epithelial electrical resistance, and/or morphology, e.g. by using hematoxylin and eosin staining and/or immunofluorescence. It is further noted that a tubular shaped fluidic device is preferred for sampling cells as well as non-cellular, unicellular, multicellular organisms, tissue and/or materials and/or products and/or metabolites from the fluidic device of the present invention.

In another aspect, the present invention relates to a fluidic device as described above, wherein the first compartment is a tissue compartment comprising tissue cells, the second compartment is a circulatory system compartment comprising circulatory system cells and, optionally, the third compartment is a neural compartment comprising neuronal cells.

The one or more compartments of the present invention may (further) comprise biological, non-biological, physical, biophysical, chemical and/or biochemical stimuli.

The biological stimuli may involve, relate to, or derived from biology or living organisms, such as bacteria and immune cells, whereas the non-biological stimuli do not involve, relate to, or derived from biology or living organisms. The physical stimuli may include the use of ultraviolet light, pressure, temperature and combinations thereof, whereas the biophysical stimuli refers to the stimulation/activation of cell membranes, e.g. by using agonists or antagonists compounds interacting with the receptors comprised in the cell membranes, by altering the electric potential of the membrane. The chemical stimuli may be selected from active pharmaceutical ingredients or natural active agents, such as oxygen or nitric oxide, whereas the biochemical stimuli may be selected from chemical agents derived from biology or living organisms, such as cytokines.

In an embodiment, the inner and/or outer surface of one or more compartments is at least partially coated with a layer of cells selected from tissue cells, circulatory system cells, optionally, neuronal cells and combinations thereof. In a favourable embodiment, the first compartment, e.g. the tissue compartment is at least partially coated with a layer of tissue cells. In another favourable embodiment, the second compartment, e.g. the circulatory system compartment is at least partially coated with a layer of circulatory system cells preferably forming a capillary endothelium. Such capillary endothelium may be formed by coating the entire inner and/or outer surface of the circulatory system compartment with a layer of circulatory system cells or by the formation of a capillary endothelium by circulatory system cells within the circulatory system compartment itself. The capillary endothelium may be formed by coating the outer surface of the circulatory system compartment made by a biodegradable material with blood and/or microvascular cells. Optionally, also the inner and/or outer surface of the third compartment, e.g. the neural compartment may be at least partially coated with a layer of neuronal cells.

In an embodiment of the present invention, at least a part of the at least partially coated inner and/or outer surface of one of the compartments is contiguous to at least a part of the inner and/or outer surface of the at least two other compartments. In this context the term “contiguous” has to be understood that the inner and/or outer surfaces of the different compartments share a common border, e.g. the separating materials optionally including the interstitial space enclosed by the outer surfaces of the compartments.

In an even further embodiment of the present invention, at least a part of the at least partially coated inner and/or outer surface of the one or more compartments may further comprise a layer of connective tissue. Preferably the connective tissue is located in between the inner and/or outer surface of at least one of the compartments and the layer of cells selected from tissue cells, circulatory system cells and, optionally, neuronal cells. The layer of connective tissue may comprise ECM, interstitial cells, products and/or metabolites. The connective tissue may further be chosen such that the layer of connective tissue has adhesive properties, e.g. by using fibroblasts, to adhere cells selected from tissue cells, circulatory system cells and neuronal cells to the inner and/or outer surface of the compartment and/or to the area comprising a plurality of pores, e.g. the above-described permeable and/or semi-permeable material, e.g. permeable and/or semi-permeable membrane.

Other adhesive materials may be used as well to adhere cells selected from tissue cells, circulatory system cells and, optionally, neuronal cells to the inner and/or outer surface of one or more compartments. Preferably the material used to adhere cells to the inner and/or outer surface of one or more compartments is selected from a biocompatible material. The adhesive material is preferably applied to the inner and/or outer surface of the channel as a gel, solution, hydrogel, micro-carrier beads, self-assembling micro- and nanofluidic devices or other composition that will adhere to the inner and/or outer surface of the compartment via or without binding to the material of which the surface of the compartment is made of.

In an embodiment of the present invention, the adhesive material is chemically coupled to the inner and/or outer surface of the compartment, e.g. via a covalently bond or cross-link. In another embodiment, the membrane comprised in the separating material is created (e.g. polymerized) with adhesive material embedded in the membrane. In even another embodiment, the adhesive material can be a molecule bound by a molecule on the surface of a tissue cell. In even a further embodiment, the adhesive material can be a molecule which binds a molecule on the surface of the tissue cell.

Preferably the adhesive coating material is selected but not limited from the group consisting of collagen, laminin, proteoglycan, vitronectin, fibronectin, fibrin, poly-D-lysine, elastin, hyaluronic acid, glycoasaminoglycans, integrin, polypeptides, oligonucleotides, DNA, polysaccharide, MATRIGEL™, extracellular matrix, synthetic and/or natural self-assembling gels, e.g. self-assembling peptide Puramatrix™ and combinations thereof.

In an embodiment of the present invention, the adhesive material may be obtained from a mammal or synthesized or obtained from a transgenic organism. Preferably, the adhesive material is mammalian, e.g. murine, primate or human in origin. Furthermore, the concentration of the adhesive material may vary. Preferably, the adhesive material is present at a concentration in range from about 10 μg/mL to about 1000 μg/mL, more preferably present in an amount of 10 μg/mL, 50 μg/mL, 100 μg/mL, 200 μg/mL, 300 μg/mL, 500 μg/mL, 1000 μg/mL or any value in between.

In a particular embodiment of the present invention, the separating material of the fluidic device separating the compartments comprised in the at least one set of distinct compartments may be coated with a mixture comprising collagen type I, preferably, the separating material may be coated with 400 μg/mL collagen type I. In another embodiment of the present invention, the separating material is coated with a mixture comprising 0.1 U/mL thrombin and 2 mg/mL fibrinogen optionally dissolved in a desired cell culture medium.

In a further embodiment of the present invention, the at least one of the compartments, e.g. the tissue, circulatory system or neural compartment, and/or the interstitial space of the fluidic device of the present invention comprises at least one hollow membrane for culturing and/or co-culturing, evaluating, sampling and/or harvesting of tissue cells, circulatory system cells, neuronal cells, interstitial cells, products and/or metabolites from the fluidic device.

As used herein, the term “hollow membrane” refers to any fibre, capillary, channel, tube, or groove that is deposed within or upon a substrate. The hollow membrane can be a microchannel, i.e. a fibre that is sized for passing through microvolumes of liquid.

Preferably the at least one hollow membrane is embedded in at least one of the coatings formed on the inner surface of one or more compartments. Favourably, the hollow membrane is made of a permeable and/or semi-permeable material, e.g. permeable and/or semi-permeable membrane. Even further, the hollow membrane is at least partially made of a biodegradable material. The porosity of hollow membrane material and/or MWCO depend on specific needs and the maximum molecular weight of the desired dissolved compound and/or a cell that will pass through the permeable and/or semi-permeable membrane into the permeate stream. Since the permeability of the hollow membrane may be varied per surface area of the hollow membrane, the scientists/bioengineers have the possibility to design the hollow membrane in such a way that any kind of components can be administered to a specific part of the fluidic device by using the hollow membrane. Consequently, the permeability of the hollow membrane may be chosen such that samples can be taken from the interstitial space or tissue, circulatory system or neural compartments depending on the location of the hollow membrane. The usage of hollow membranes located in one of the compartments or embedded in the coatings as described above, allows the (dynamic) sampling extracellular fluids (e.g. interstitial fluid), tissue, circulatory system or neural cells to evaluate cellular characteristics like proteomics and metabolomics to provide a more complete picture of a living organism.

The separating material separating the at least three compartments, fluid channel and/or hollow membrane of the present invention may have different thickness. Preferably the separating material of the fluidic device separating the at least three compartments, fluid channel and/or hollow membrane is from 0.5 μm or greater in thickness, favourably 5 μm or greater in thickness, preferably 10 μm or greater in thickness, more preferably 20 μm or greater in thickness, 25 μm or greater in thickness, 30 μm or greater in thickness, 35 μm or greater in thickness or 40 μm or greater in thickness. Favourably, the separating material of the fluidic device separating the at least three compartments, fluid channel and/or hollow membrane have a thickness in the range from about 10 μm to about 50 μm.

At least a part of the separating material of the fluidic device separating the at least three compartments, fluid channel and/or hollow membrane is made of a biocompatible polymer wherein biocompatible polymer refers to materials which do not have toxic or injurious effects on biological functions. Biocompatible polymers may include but are not limited to natural, ECM derived compounds like collagen, laminin, Matrigel™ or the like or synthetic biodegradable or non-biodegradable polymers, e.g. poly(alpha esters) such as poly (lactate acid), poly(glycolic acid), polyorthoesters and poly anhydrides and their copolymers, polyglycolic acid and polyglactin, cellulose ether, cellulose, cellulosic ester, fluorinated polyethylene, phenolic, photoresist, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, poly ether sulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, self-assembling peptides such as Puramatrix™, self-assembling organic and/or inorganic micro- and nanofluidic devices such as parylene microplates, urea-formaldehyde, polyglactin, or copolymers or physical blends of these materials.

At least a part of the separating material of the fluidic device separating the at least three compartments, fluid channel and/or hollow membrane may also be made of, for example, ceramic coatings on a metallic substrate. However, any type of coating material may be suitable. The coating may be made of different types of materials including metals, ceramics, polymers, hydrogels or a combination of any of these materials. Biocompatible materials may include, but are not limited to an oxide, a phosphate, a carbonate, a nitride or a carbonitride. The oxide may be selected from the group consisting of tantalum oxide, aluminum oxide, iridium oxide, zirconium oxide or titanium oxide.

The present invention further relates to the use of the fluidic device of the present invention for culturing, co-culturing, evaluating, sampling and/or harvesting of tissue cells, circulatory system cells, neuronal cells, interstitial cells, products and/or metabolites from the fluidic device. The at least one set of distinct compartments, i.e. the tissue compartment, the circulatory system compartment and, optionally, the neural compartment, may be used to culture, coculture, evaluate, sample and/or harvest cell material and/or fluids. The fluidic device therefore allows the scientist/bioengineer to culture, coculture, evaluate, sample and/or harvest in vitro reconstructed tissue and to study possible relevant therapies for patient specific tissues.

The present invention also relates to the use of the fluidic device of the present invention for culturing, co-culturing, evaluating, sampling and/or harvesting of non-cellular, unicellular and/or multicellular organisms and/or tissue, material, products and/or metabolites from the fluidic device other than the reconstructed tissue.

In another aspect the present invention relates to a perfusion system, e.g. a bioreactor, comprising at least one fluidic device as described above. In a preferred embodiment the perfusion system comprises at least one first, at least one second and at least one third inlet port each inlet port being arranged for feeding medium to fluidic device and at least one first, at least one second and at least one third outlet port outlet port being arranged for discharging medium from the fluidic device, wherein the at least one first inlet and outlet port are connected to the at least one tissue compartment, the at least one second inlet and outlet port are connected to the at least one circulatory system compartment and the at least one third inlet and outlet port are connected to the at least one neural compartment.

The term “port” refers to a portion of the perfusion system described herein which provides a means for fluid and/or cells to enter and/or exit the system and/or to enter and/or exit portions of the system. The port can be of any size and shape to accept and/or secure a connection with tubes, connections, or adaptors of a fluidic or microfluidic system and allow passage of fluid and/or cells when the port is attached to a fluidic or microfluidic system.

The perfusions system of the present invention may further comprise at least fourth inlet port for feeding medium to the fluidic device and at least one fourth outlet port for discharging medium from the fluidic device, wherein the at least one fourth inlet and outlet port are connected to the at least one interstitial space of the fluidic device. Preferably, the fourth inlet and outlet port are arranged for feeding and discharging interstitial cells and/or fluid to the fluidic device of the present invention.

In case the fluidic device is provided with hollow membranes to evaluate and/or sample tissue cells, circulatory system cells, neuronal cells, interstitial cells and/or products and/or metabolites from the fluidic device of the present invention, the fluidic device of the present invention may further comprise at least one fluid inlet port and at least one fluid outlet port connected to the hollow membrane of the fluidic device.

In a further embodiment, in case the fluidic device of the present invention, e.g. tubular shaped fluidic device, provides a fourth compartment comprising one or more fluid compartments, e.g. interstitial fluid compartments, the fourth inlet and outlet port of the fluidic device may be connected with the one or more fluid compartments. The fluidic device may further comprises at least one fifth inlet port for feeding medium to the fluidic device and at least one fifth outlet port for discharging medium from the fluidic device, wherein the at least one fifth inlet port and at least one fifth outlet port may be connected to the remaining external space of the fluidic device, wherein the external space is the space enclosed by the interior of the fluidic device and the outer surfaces of the separated compartments (optionally in combination with interstitial, tissue, circulatory system and/or neural cells separating the external space from the interstitial space).

In an even further embodiment, the perfusion system of the present invention comprises a fluidic device comprising two or more sets of separated compartments and wherein the at least one first inlet and outlet port are connected to two or more tissue compartments, the at least one second inlet and outlet port are connected to two or more circulatory system compartments and the at least one third inlet and outlet port are connected to two or more neural compartments.

Additionally, the inlet ports arranged in the perfusion system of the present invention may further comprise one or more sample inlet ports allowing the scientist/bioengineer to administer any kind of component, e.g. cell material, microbial cells, pathogens, parasites, pharmaceutically active ingredients, signalling molecules, growth factors, hormones or the like to the fluidic device of the present invention. The fluidic device of the present invention may further comprise one or more sample outlet ports allowing the scientist/bioengineer to collect samples, e.g. cells, products and/or metabolites, products of coculture with other than reconstructed desired tissue non-cellular, unicellular and/or multicellular organism and/or tissue, material, products and/or metabolites or the like, from the fluids discharged from the compartments of the fluidic device of the present invention.

As already mentioned above, physical, chemical and/or biological stimuli, e.g. irradiation, light, gas, cell material, microbial cells, pathogens, parasitic and/or symbiotic organism, pharmaceutically active ingredients, signalling molecules, growth factors, hormones or the like, may be used to evaluate responses of a constructed living tissue to desired stimuli and/or to evaluate responses of applied stimuli to constructed tissue. The above-mentioned stimuli may be applied and/or administered by the scientist/bioengineer to the fluidic device of the present invention wherein the constructed and maintained living tissue and/or cocultured guest organism, tissue and/or material in the fluidic device are exposed to the desired stimuli for a predefined period of time. For example, to stimulate the natural reconstruction of intestinal epithelial cells, microbial cells may be maintained in the fluidic device of the present invention for at least 1 day.

The above-mentioned pharmaceutically active ingredients, signalling molecules, growth factors, hormones or the like may be selected from the group consisting of therapeutics, small molecules, nutriceuticals, drugs, probiotics, foods, vitamins, food supplements, commensal and pathogenic microflora, toxins and combinations thereof.

The biological stimuli may be non-cellular and/or cellular, unicellular and/or multicellular, aerobic and/or anaerobic and the fluidic device of the present invention may comprise a combination. Even further, to stimulate the natural growth of tissue cells, e.g. gut, intestinal microbiota are preferably supplied to the tissue compartment of the fluidic device of the present invention.

It is noted that the fluidic device and/or perfusion system of the present invention allows the scientist/bioengineer to coculture the in vitro constructed tissue with another organism and/or tissue, wherein the other tissue is not necessarily constructed in vitro. Even further, the tissue, circulatory system, and/or neuronal cells comprised in the tissue, circulatory system and neural compartments may be cocultured with other organisms, tissues and/or materials. For example, coculturing intestinal epithelium and intestinal microbiota in the tissue compartment may be used to study host microbe interactions and/or to culture difficult to culture intestinal microbiota. The circulatory system cells in the circulatory system compartment may be cocultured with the Plasmodium malaria and the neuronal cells may be cocultured with the poliovirus to study host pathogen interactions. Even further, connective tissue applied to the compartments may be combined with other tissues and/or organisms as well. For example, the connective tissue may be combined with Echinococcus. In an even further aspect the fluidic device of the present invention allows to use the reconstructed tissue as a feeding and/or support tissue, e.g. to study in utero embryonic development. The fluidic device and/or perfusion system of the present invention further allow the scientist/bioengineer to coculture human and/or animal tissue with other than reconstructed tissue non-cellular, unicellular and multicellular organisms and/or tissue and/or material, e.g. biomedical polymers and/or donor tissues to study transplantation rejection.

In a further aspect the present invention relates to a perfusion system of the present invention further comprising at least one first, at least one second and at least one third reservoir coupled to the at least one first, at least one second and at least one third inlet ports of the fluidic device for feeding medium to the fluidic device. The reservoir may be selected from a pressure resistant reservoir or other container comprising a medium such as a fluid (e.g. water or tissue specific medium) or a gas (e.g. air, pressurized gas and/or other gas).

The reservoir can be a container comprising a volume of fluid such that the fluid can be caused to move from the reservoir and through the one or more compartments of the fluidic device. The reservoir can be coupled to the one or more fluidic devices of the perfusion system by any means of conducting a fluid, e.g. tubing, piping, compartments, or the like. The fluidic device and/or the reservoir can comprise ports. The reservoir may also be a syringe connected to the fluidic device of the present invention. The use of a syringe allows the scientist/bioengineer to add and/or sample products and/or metabolites from the fluidic device, e.g. interstitial fluid, without a permanent flow of fluid through the respective compartment, e.g. separated compartment, interstitial space or external space.

The medium which is caused to flow through the one or more compartments, fluid compartments and/or hollow membranes of the fluidic device described herein may be any medium appropriate for maintaining or culturing tissue cells, circulatory system cells, neuronal cells and/or interstitial cells. The medium flow through the different compartments, fluid compartments and/or hollow membranes may be substantially the same medium or may vary per part of the fluidic device of the present invention. In a preferred embodiment of the present invention, the medium flow through the different compartments, fluid compartments and/or hollow membranes is substantially different from one another. In case microbial cells are present in the fluidic device, the medium should be appropriate for maintaining or culturing microbial cells, preferably the medium should not contain antibiotics to which the microbial cells are susceptible. The medium may comprise cell culture medium, solutions, buffers, nutrients, tracer compounds, dyes, antimicrobials, or other compounds not toxic to the cells being cultured in the fluidic device described herein. Suitable media for culturing or maintaining tissue cells, e.g. intestinal cells, intestinal epithelial cells, endothelial cells, immune cells, and/or connective tissue cells, and microbial cells are well known in the art. By way of non-limiting example, media suitable for maintaining or culturing tissue cells, e.g. intestinal epithelial cells can include Advanced DMEM/F12 Medium (Invitrogen) containing BSA (Sigma) supplemented with EGF, R-spondin 1 and Noggin growth factors (Peprotech), penicillin, streptomycin (Gibco) and/or Normocin (Invivogen, San Diego, Calif.).

The at least one first, at least one second and at least one third reservoir may be coupled to the at least one first, at least one second and at least one third outlet ports respectively for receiving medium from the fluidic device. By connecting the outlet ports of the fluidic device with the at least three reservoirs a closed system can be created in order to reduce any negative influence from the surrounding environment. As already explained above, such closed system may be provided with one or more sample inlet and/or sample outlet ports to allow the scientist/bioengineer to influence the system in a controllable way. In order to provide a constant flow of medium, the perfusion system of the present invention may further comprise at least one pump coupled to the at least one fluidic device and the at least one first, at least one second and/or at least one third reservoirs. It is noted that further ports, e.g. the fourth and fifth port, may be connected to a pump as well. Even further, as already mentioned above, the ports may be connected to a syringe.

The at least one pump may be any dynamic or displacement pump and may be selected from the group consisting of a syringe pump, a peristaltic pump, pulse-free pump, positive displacement pump and combinations thereof.

The flow of the medium through the fluidic device is capable to generate well-defined wall shear stress that affects cellular morphology and physiology, e.g. genomics, transcriptomics, proteomics and/or metabolomics. Biomechanical stimulation of physiological magnitude can modulate cellular phenotype via modulation of gene expression. As already explained above, the fluidic device of the present invention can be planar. The flow shear stress (τ) at the wall of the compartments contained in a planar fluidic device is a function of flow rate and height of the compartment. The shear stress on the cells is assumed approximately equal to the compartment wall in case the cell height is approximately two orders of magnitude less than the compartment. Equation 1 describes the relationship between the shear stress and the flow rate in a planar fluidic device.

τ=6Qμ/(wh²) (1)

wherein:
τ is the shear stress in dyne/cm²;
Q is the flow rate in cm³/s;
μ is the dynamic viscosity of the culture medium in g/cm·s;
w is the flow compartment width in cm; and
h is the flow compartment height in cm.

The compartments contained in the fluidic device of the present invention can also have a tubular form. In a tubular shaped compartment the wall shear stress in the circumferential direction on the inner surface of the compartment wall/cells can be described by equation 2.

τ=4μQ/πr³ (2)

wherein:
τ is the shear stress in dyne/cm²;
μ is the dynamic viscosity of the culture medium in g/cm·s;
Q is the volume flow rate in cm³/sec;
π is the known mathematical constant; and
r is the radius in cm.

The shear stress on the medium flowing through the fluidic device compartments may be from 0 to 1000 dyne/cm². Preferably, the shear stress can be in the range from about 0.5 dyne/cm² to about 120 dyne/cm². The shear stress and/or the flow rate can be modulated to create a desired state and/or condition of the living tissue cells, such as intestinal epithelial cells, e.g. modelling “flush-out” of the luminal components of the intestine.

The shear stress may be about the same for the duration of the time during which living cells are cultured in the fluidic device. However, in an embodiment of the present invention, the shear stress may be increased and/or decreased during the time in which living cells are cultured in the fluidic device, e.g. the shear stress may be decreased for a time to allow newly added cells to attach to the membrane and/or pre-existing cells. Preferably, the shear stress may be varied in a regular, cyclic pattern to mimic desired tissue deformation, e.g. blood vessels pulsation. On the other hand, in another embodiment of the present invention, the shear stress can be varied in an irregular pattern, e.g. mimic intestinal motility. The shear stress of the medium flowing through the fluid compartment on the cells presented in the flow compartment can vary over time. In an embodiment of the present invention, the shear stress can vary over time from 0 to 1000 dyne/cm². In a particular embodiment of the present invention, the shear stress can vary over time from 0.5 dyne/cm² to 34 dyne/cm².

Different flow rates of the medium through the compartments of the fluidic device may be applied to the perfusion system of the present invention. The flow rate may be varied between the different compartments and may be varied in such way to mimic the in vivo flow rate of a flow through the desired living tissue. Even so, the flow rate of the medium can be adjusted to mimic the flow of a medium in case the living tissue is suffering from a disorder affecting the respective living tissue constructed in the in vitro system of the present invention, e.g. to mimic diarrhoea.

The flow rate may be varied over time. In an embodiment of the present invention, the medium flow rate may be about the same for the duration of the time during which living cells are cultured in the fluidic device of the present invention. In a particular embodiment, the medium flow rate can be increased and/or decreased during the time in which living cells are cultured in the fluidic device, e.g. the medium flow rate can be decreased for a time to allow newly added cells to attach to the membrane and/or pre-existing cells. Alternatively, the medium flow rate can be varied in a regular, cyclic pattern or in an irregular pattern.

The perfusion system of the present invention may further comprise units for monitoring and controlling several process parameters, including the pH value, pressure, flow rate, temperature and the like. The perfusion system of the present invention may further comprise filters and/or an oxygenator.

In another aspect the present invention relates to a method for in vitro culturing and/or co-culturing cells, including complex living tissue reconstruction, comprising the following steps:

a) providing a perfusion system of the present invention;
b) providing tissue cells, circulatory system cells and, optionally, neuronal cells;
c) allowing medium to flow through the fluidic device;
d) closing the inlet ports and outlet ports of the fluidic device to stop the flow of medium once the fluidic device is filled with medium;
e) seeding the tissue cells to the first compartment of the fluidic device;
f) seeding the circulatory system cells to the second compartment of the fluidic device;
g) optionally, seeding the neuronal cells to the third compartment of the fluidic device; and
h) open the inlet ports and outlet ports of the fluidic device to allow medium to flow through the fluidic device.

The above-described method can be applied for any type of fluidic device. In an embodiment of the present invention, the method further comprises the steps of providing a connective tissue and coating the inner and/or outer surface of the first, second and/or third compartment with the connective tissue before seeding the tissue cells, the circulatory system cells and/or neuronal cells to the respective compartments.

The separating material of the fluidic device separating at least a part of the at least three different compartments may be pre-coated with connective tissue before placing the separating material, e.g. permeable and/or semi-permeable membrane, into the fluidic device of the present invention. Even further, tissue cells, the circulatory system cells and/or neuronal cells may be seeded to the separating material separating at least a part of the at least three different compartments before placing the separating material into the fluidic device of the present invention.

Alternatively the present invention relates to a method for in vitro culturing and/or co-culturing cells, including complex living tissue reconstruction, comprising the following steps:

a) providing at least three separating materials for forming at least three physically separated channels;
b) providing tissue cells, circulatory system cells and, optionally, neuronal cells;
c) seeding each of the tissue cells, circulatory system cells and neuronal cells onto the inner and/or outer surface of one of the at least three separating materials;
d) placing the seeded at least three separating materials into a fluidic device of to the present invention;
e) connecting the fluidic device to a perfusion system; and
f) allowing medium to flow through the fluidic device,
wherein the method further comprises applying biological, non-biological, physical, biophysical, chemical and/or biochemical stimuli to one or more of the channels.

The physically separated separating materials may be formed such that planar or tubular fluid channels are created. Again, connective tissue may be provided to the wall of the separating materials before seeding tissue cells, circulatory system cells and neuronal cells onto the material.

The separating material may be pre-coated with an adhesive, e.g. collagen type I, to enhance the cell adhesion to the separating material. The separating material may be selected but not limited from the group consisting of collagen, laminin, proteoglycan, vitronectin, fibronectin, poly-D-lysine, elastin, hyaluronic acid, glycoasaminoglycans, integrin, polypeptides, oligonucleotides, DNA, polysaccharide, MATRIGEL™, Puramatrix™, extracellular matrix, self-assembling micro- and nanofluidic devices such as parylene microplates and combinations thereof.

In an even further aspect the present invention relates to a hollow membrane for evaluating, sampling and/or harvesting of tissue cells, circulatory system cells, neuronal cells, interstitial cells, products, products and/or metabolites from the fluidic device of the present invention, wherein the hollow membrane is made of a permeable and/or semi-permeable material, e.g. permeable and/or semi-permeable membrane. The hollow membranes are in particular suitable to meet scientific and industrial needs to allow scientists/bioengineers to control, evaluate, sample and/or harvest any characteristic of the in vitro reconstructed tissue. Preferably, the membrane of the hollow membrane is made of regenerated hydrophilic and/or hydrophobic, coated and/or uncoated biocompatible material for a long-term cell culture system. The material of the membrane may be selected but not limited from the group consisting of cellulose, cellophane, polyethylene, silicone, carbon nanomembranes and combinations thereof.

In a final aspect the present invention relates to the use of the hollow membrane as described above for culturing, co-culturing, evaluating, sampling and/or harvesting of tissue cells, circulatory system cells, neuronal cells, interstitial cells, products and/or metabolites.

The invention will be elucidated on the basis of non-limitative exemplary embodiments shown in the following figures, in which:

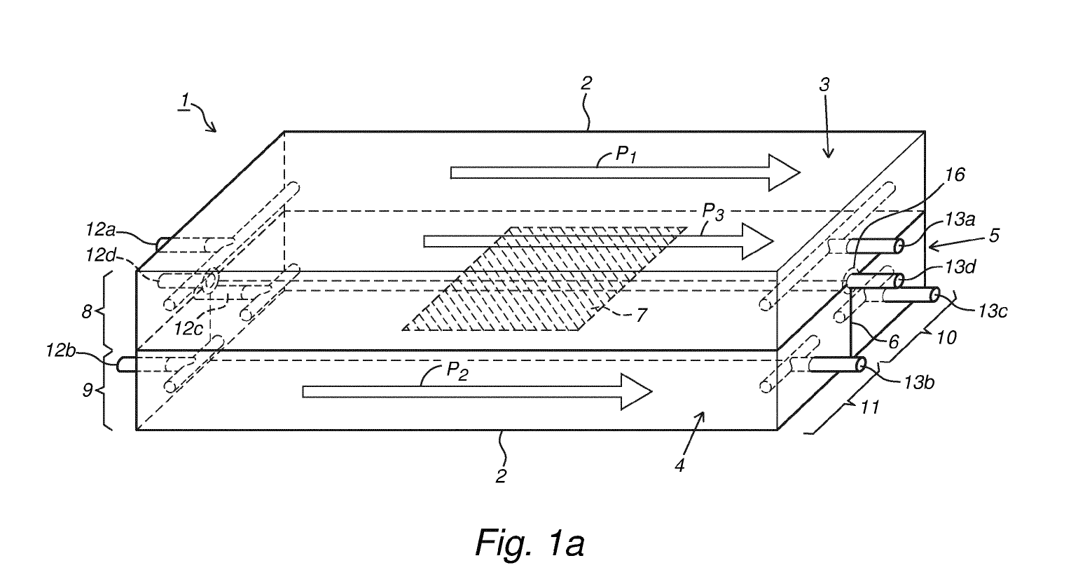

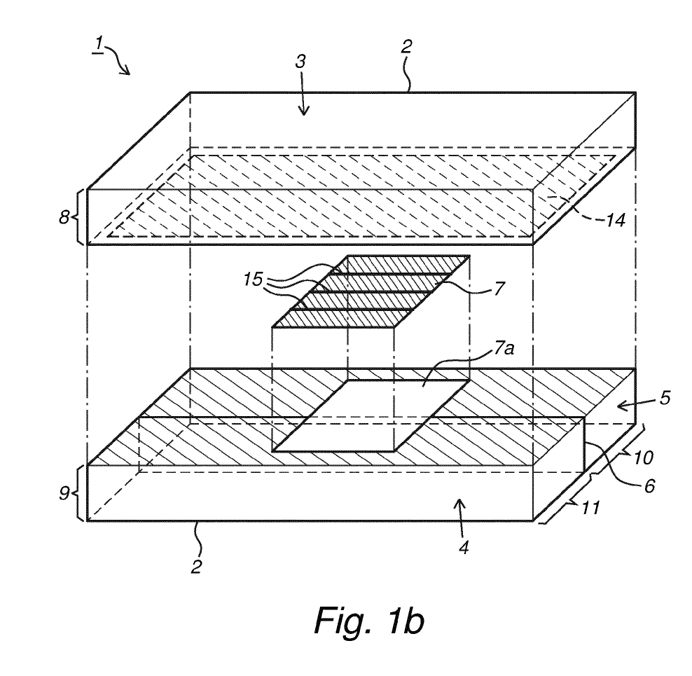

FIG. 1a shows a schematic view of a planar fluidic device for in vitro complex living tissue reconstruction according to the present invention;

FIG. 1b shows an exploded view of a planar fluidic device for in vitro complex living tissue reconstruction according to the present invention;

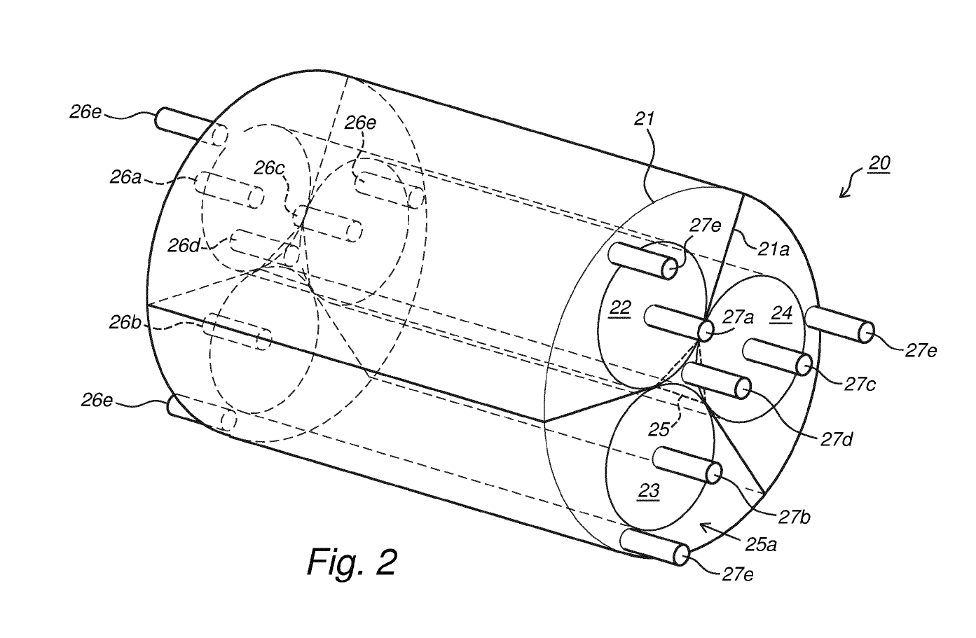

FIG. 2 shows a schematic view of a tubular fluidic device for in vitro complex living tissue reconstruction according to the present invention;

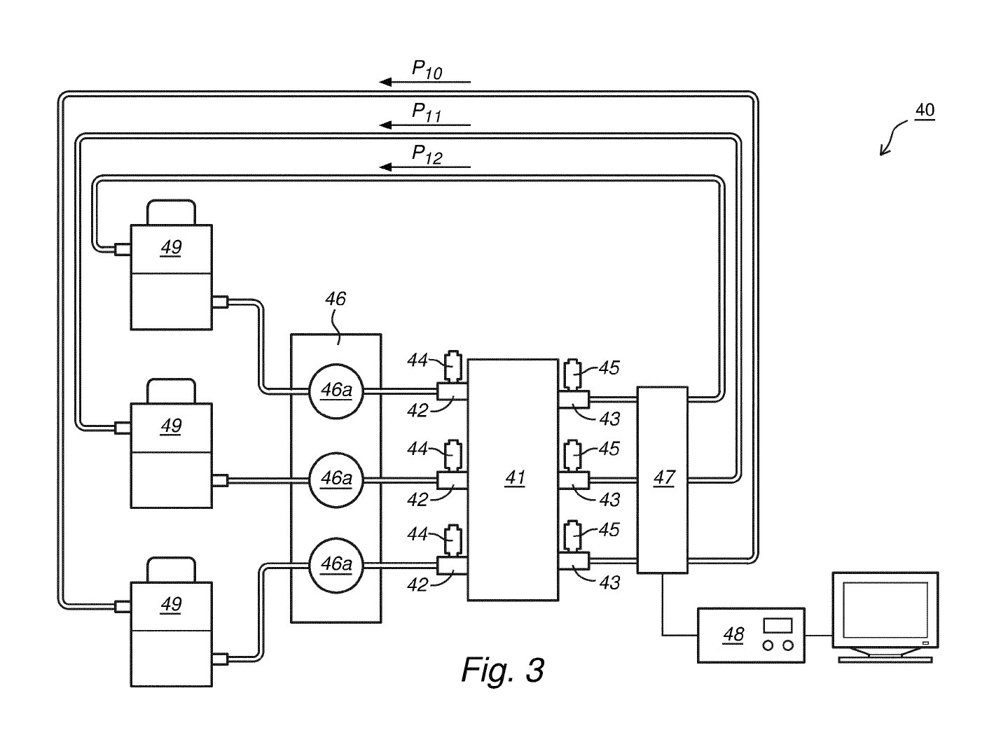

FIG. 3 shows a schematic view of a perfusion system comprising the fluidic device for in vitro complex living tissue reconstruction according to the present invention;

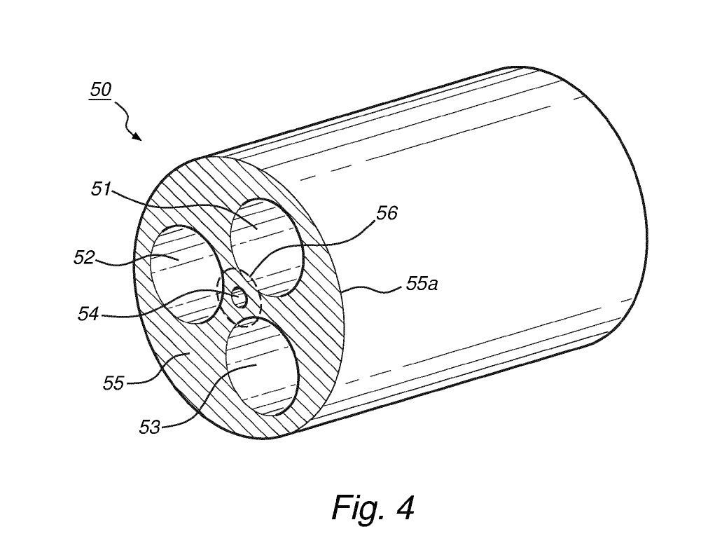

FIG. 4 shows a schematic view of a further tubular fluidic device for in vitro tissue reconstruction according to the present invention; and

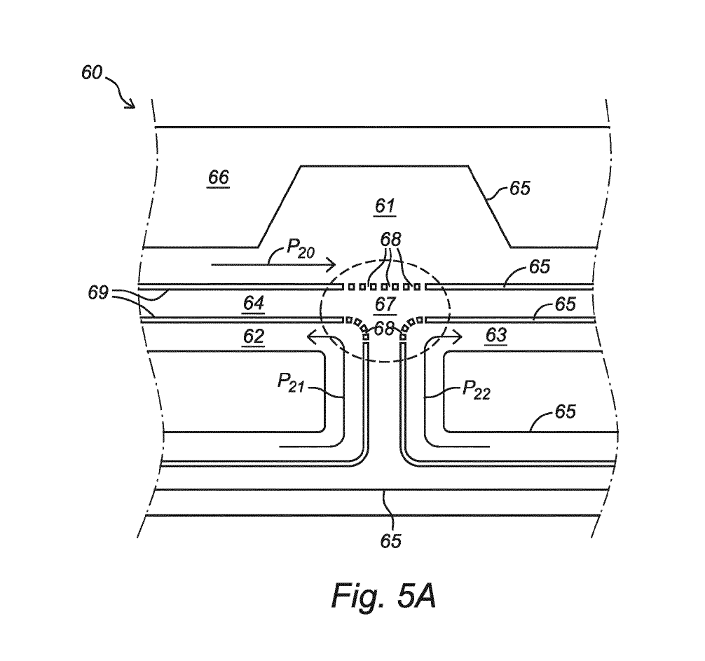

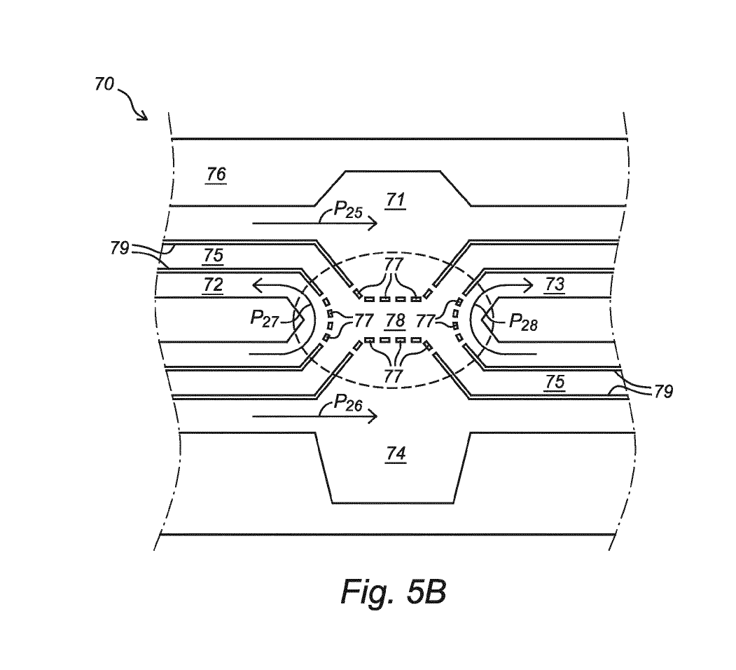

FIGS. 5a and 5b show a schematic top view of a planar fluidic device for in vitro complex living tissue reconstruction according to the present invention.

FIG. 1a shows a schematic view of a planar fluidic device 1. The planar fluidic device 1 comprises an interior 2 comprising a first channel, i.e. upper flow channel 3, a second channel, i.e. lower left flow channel 4, and a third channel, i.e. lower right flow channel 5. The three different channels 3, 4, 5 are separated from one another by T-shaped separating portion 6 and the three different channels 3, 4, 5 congregate with one another in exchange region 16. The separating portion 6 may also have a different form than illustrated, e.g. Y-shaped, as long as the separating portion 6 separates the three different channels 3, 4, 5. Each of the channels 3, 4, 5 of FIG. 1a is enclosed by a part of the interior 2 and a part of the separating portion 6. It is noted that the channels 3, 4, 5 may be enclosed entirely by the separating portion 6 (see in this respect: FIG. 2). Even so, the separating portion 6 may be made of physically separated materials wherein the outer surfaces of the physically separated materials enclose a space (not shown) situated in between the different channels 3, 4, 5. The separating portion 6 further comprises a membrane 7 for culturing tissue cells, circulatory system cells and/or neuronal cells each seeded to a part of the membrane 7 facing the channels 3, 4, 5. The membrane 7 physically separates the three flow channels 3, 4, 5 from one another, but allows cells cultured on the membrane 7 to communicate with each other. The membrane 7 may be a matrix whereon and/or wherein the cells can be seeded. The membrane 7 is preferably provided with (a layer of) hollow membranes 15 placed on and/or into the membrane 7 for evaluating, sampling and/or harvesting cells and/or fluid from the interstitial space (see in this respect: FIG. 1b). Favourably, the membrane 7 and hollow membranes are assembled as an insert but may also be directly incorporated into the fluidic device. Optionally, the separating portion 6 may consist entirely of a semi-permeable and/or permeable membrane, e.g. the membrane 7 as illustrated. It is noted that the material of the separating portion 6 dividing the interior 2 of the fluidic device 1 into an upper part 8 and a lower part 9 may be different from the material of the separating portion 6 dividing the lower part 9 into a lower right part 10 and a lower left part 11. It is further noted that the arrangement of the channels 3, 4, 5 may be completely different from the arrangement of the channels 3, 4, 5 illustrated in FIG. 1a, as long as the three different channels 3, 4, 5 are physically separated by a separating portion 6, which separating portion 6 comprises means, such as pores (not shown) or a membrane 7, allowing communication between each of the channels 3, 4, 5. The fluidic device 1 of FIG. 1a further comprises inlet ports 12a, 12b, 12c and outlet ports 13a, 13b, 13c to allow medium to flow through the different channels in a direction illustrated by arrows P₁, P₂. P₃. The fluidic device 1 of FIG. 1a further comprises an inlet port 12d and outlet port 13d connected to the hollow membrane (not shown) for evaluating, sampling and/or harvesting cells and/or fluid of the fluidic device. Optionally, each hollow membrane may be connected to a separate inlet and/or outlet port (not shown) to separate interstitial fluid and/or cells from different areas of living tissue. FIG. 1a depicts a fluidic device 1 wherein the flow of medium in each channel 3, 4, 5 is parallel to one another (see: arrows P₁, P₂. P₃). However, the flow of medium in one channel may be in opposite direction compared to the direction of the flow of medium in another channel. Furthermore, the type flow of medium may differ between the different channels 3, 4, 5, e.g. the flow in one channel may be laminar where the flow in another channel may be turbulent.

FIG. 1b shows an exploded view of the planar fluidic device 1. It is noted that the fluidic device 1 is in general preferably made from transparent material to allow a visual control of the in vitro model. FIG. 1b shows the upper part 8 comprising the interior 2 and first channel 3. The first channel 3 is provided with an opening 14. FIG. 1b further shows the lower part 9 comprising a lower right part 10 and a lower left part 11 separated by T-shape separating portion 6. Channels 4, 5 are enclosed by the interior 2 and separating portion 6. Separating portion 6 is provided with an opening 7a. FIG. 1b further shows an insert with membrane 7 which fits the membrane 7 onto the opening 7a provided in separating portion 6. The fluidic device 1 is assembled by attaching membrane 7, whether or not seeded with cells, onto opening 7a and subsequently attaching upper part 8 to lower part 9.

FIG. 2 shows a schematic view of a tubular fluidic device 20 comprising an interior 21 comprising a first channel, i.e. tubular shaped flow channel 22, a second channel, i.e. tubular shaped flow channel 23, and a third channel, i.e. tubular shaped flow channel 24. Each tubular shaped flow channel 22, 23, 24 is preferably formed by a membrane having a certain degree of permeability to allow communication of cells contained in each of the tubular shaped flow channel 22, 23, 24. In FIG. 2, the tubular shaped flow channels 22, 23, 24 are located adjacent to each other in the exchange region, to enclose an interstitial space 25 separated from external space 25a enclosed by the inner surface of the interior 21 and the outer surface of the tubular shaped flow channels 22, 23, 24. The interstitial space 25 may be arranged to receive interstitial fluid and/or interstitial cells. It is noted that the adjoining of flow channels 22, 23, 24 is not necessary to allow communication between the different channels 22, 23, 24. The different flow channels 22, 23, 24 may be placed at a distance from one another. The interstitial space 25 enclosed by the outer surfaces of the flow channels 22, 23, 24 may be presented by an interstitial fluid channel formed by the outer surfaces of the flow channels 22, 23, 24, which flow channels are in communication with the interstitial fluid channel. The use of such natural occurred interstitial channel is preferred to provide sampling interstitial fluid for separation and/or purification of desired compounds and/or products and/or metabolites using separation and/or purification technology, e.g. liquid chromatography. The external space 25a may comprise supernatant from the different cells seeded to each of the channels 22, 23, 24. It is noted that the supernatant from the different channels 22, 23, 24 may also be separated using separating portions 21a defining an external space 25a divided into different compartments enclosed by the outer surface of one of the flow channels 22, 23, 24 the inner surface of the interior 21 and the inner surface of separating portions 21a. The fluidic device 20 further comprises inlet ports 26a, 26b, 26c (not visible), 26d (not visible), 26e and outlet ports 27a, 27b, 27c, 27d, 27e, each of the inlet ports 26a, 26b, 26c, 26d, 26e and outlet ports 27a, 27b, 27c, 27d, 27e are connected to respectively one of the channels 22, 23, 24, the interstitial space 25 and the external space 25a of the fluidic device 20. It is noted that the inlet port 26d and outlet port 27d may be connected to a hollow membrane (not shown) which hollow membrane is in communication with each of the channels 22, 23, 24. In other words, the interstitial space 25 may include a plurality of hollow membranes wherein each of the hollow membranes is in close communication with the at least three channels 22, 23, 24.

It is further noted that both FIGS. 1 and 2 depicts a schematic view of a fluidic device 1, 20 wherein one set consisting of at least three channels 3, 4, 5, 22, 23, 24, and at least one interstitial space 25 is illustrated. It should be understood that the cell fluidic device 1, 20 of FIGS. 1 and 2 may comprise a plurality of sets consisting of at least three channels 3, 4, 5, 22, 23, 24, and at least one interstitial space 25. Also, the fluidic device 1, 20 of FIGS. 1 and 2 may comprise more than one interior 2, 21 each of the interiors comprising at least one set of at least three channels 3, 4, 5, 22, 23, 24.

FIG. 3 shows a schematic view of a perfusion system 40. The perfusion system 40 comprises at least one fluidic device 41 of the present invention. The perfusion system 40 may also comprise additional fluidic devices (not shown). The fluidic device 41 comprises inlet ports 42 and outlet ports 43. Each of the ports 42, 43 may be provided with sample inlet ports 44 and sample outlet ports 45 to allow the scientist/bioengineer to add desired components, e.g. cells, active agents, microorganisms or the like, to the fluidic device 41 and/or to collect samples from the fluidic device 41. The inlet ports 42 are connected to a pump 46. Each of the inlet ports 42 may be connected to separate pump heads 46a to allow the scientist/bioengineer to apply different type of flow of medium to the different flow channels and/or interstitial space and/or the external space of the fluidic device 41. The outlet ports 43 may be connected to a control unit 47 which control unit 47 is arranged to control the flow of medium through the perfusion system 40 and/or each of the channels, the interstitial space and the external space (not shown) enclosed in the fluidic device 41. Preferably, the control unit 47 is connected with a computer 48. The perfusion system 40 further comprises one or more reservoirs 49, e.g. a feeding and/or collecting reservoir of medium, connected with the inlet ports 42, via the heads 46a of the pump 46, and the outlet ports 43, via control unit 47. The reservoirs 49 may comprise different media, e.g. liquid medium or gaseous medium. It is noted that the closed perfusion system 40 as illustrated in FIG. 3 may also be arranged as an open perfusion system. In such open perfusion system, the outlet ports 43 are connected to a different (collecting) reservoir (not shown). Also combinations of both systems are possible. The pump 46 is preferable selected from the group consisting of pulse-free pumps, peristaltic pumps and combinations thereof to provide a desired flow of medium. The flow of medium may be in the direction as indicated by arrows P₁₀, P₁₁, P₁₂. However, the direction of flow of medium does not necessarily have to be in parallel to one another.

FIG. 4 shows a schematic view of a further tubular fluidic device 50, i.e. a set of three separated channels around a hollow membrane-like structure. The fluidic device is made of a semi-permeable and/or permeable, biodegradable and/or non-biodegradable membrane 55, optionally provided with a semi-permeable, permeable and/or impermeable, biodegradable and/or non-biodegradable outer surface 55a. The membrane 55 is provided with four channels: a tissue channel 51, a circulatory system channel 52, a neural channel 53 and an interstitial fluid channel 54. The membrane 55 allows communication between the different channels 51, 52, 53, 54, within the exchange region 56. The membrane 55 may be made from a matrix of hollow multi-membranes. Even further, the interstitial fluid channel 54 may further comprise additional hollow membranes. It is noted that the different hollow membranes may need different inlet/outlet ports (not shown).

FIG. 5a shows a schematic top view of a part of a further planar fluidic device 60, comprising a first channel 61, a second channel 62 and a third channel 63. The fluidic device 60 further comprises a separating material 64 located between the outer surface 69 of the three channels 61, 62, 63. The fluid flow in the three channels 61, 62, 63 is visualized by arrows P₂₀, P₂₁ and P₂₂. The channels 61, 62, 63 are enclosed by an impermeable wall 65. Such a wall 65 may be created by etching channels into an impermeable material 66. The fluidic device further defines an exchange region 67 wherein passages 68 are created in the impermeable wall 65 to allow direct communication between the contents comprised in each of the channels 61, 62, 63. It is further noted that the channel of the separating material 64 may comprise a fluid, micro-carrier beads, self-assembling micro- and nanofluidic devices or a gel.

FIG. 5b shows a schematic top view of a part of an alternative planar fluidic device 70, comprising a first channel 71, a second channel 72, a third channel 73 and a fourth channel 74 separated by a separating material 75. The flow of fluids in the respective channels 71, 72, 73, 74 is indicated by arrows P₂₅, P₂₆, P₂₇ and P₂₈. Analogous to what is described above for FIG. 5a, the channels 71, 72, 73, 74 and separating material 75 (being an interstitial space enclosed by the outer surface 79 of the channels 71, 72, 73, 74) may be formed by etching an impermeable material 76. Pillars 77 may be provided in the exchange region 78 to allow direct communication between each of the channels 71, 72, 73, 74 with one another.

The invention will now be further illustrated with reference to the following example.

### EXAMPLE

Tissue cells, circulatory system cells, neuronal cells and connective tissue cells (e.g. derived from human and/or porcine) were purchased from cell banks or isolated from tissue samples using methods isolating tissue cells as described in Sato et al. (Single Lgr5 stem cells build crypt-villus structures in vitro without a mesenchymal niche; Nature Letters, 459 (2009): pp. 262-266), isolating circulatory system cells as described in Yamamoto et al. (Proliferation, differentiation, and tube formation by endothelial progenitor cells in response to shear stress; Journal of Applied Physiology, 95 (2003): pp. 2081-2088) and isolating neuronal cells as described in Bondurand et al. (Neuron and glia generating progenitors of the mammalian enteric nervous system isolated from foetal and postnatal gut cultures; Development and disease, 130 (2003): pp. 6387-6400), which methods are herewith incorporated by reference.

Reference is made to FIG. 1b wherein the different components of the planar fluidic device are shown. The membrane and the outer surfaces of the hollow membranes of an insert were coated (on both sides) with a mix of collagen I and connective tissue cells, e.g. myofibroblasts. Tissue cells were seeded onto the coated surface of the hollow membranes and the membrane facing the upper part of the fluidic device. The insert was incorporated into the opening provided in the separating portion. Subsequently, the upper part of the fluidic device was connected to the lower part of the fluidic device.

The inlet ports of the separated channels and hollow membranes were connected via a conduit with the pump of the perfusion system (see: FIG. 3). The outlet ports of the separated channels and hollow membranes were connected via a conduit with the control unit of the perfusion system. Both the pump and control unit were connected to medium reservoirs providing a medium to the fluidic device. Medium from the reservoirs was allowed to flow through the fluidic device.

Cell suspensions comprising circulatory system or neuronal cells were prepared. The pump of the perfusion system was stopped and the inlet and outlet ports of the fluidic device were closed. Syringes comprising suspensions of circulatory system and neuronal cells were connected with one of the sample inlet ports connected with the inlet port of the second or third channel, i.e. the inlet port of the lower right or lower left flow channel. The cells were loaded into the flow channels and excess of medium was removed from the flow channels of the fluidic device via the sample outlet ports using empty syringes. After removal of the syringes from the sample ports, the inlet and outlet ports were opened and the medium from the medium reservoirs was allowed to flow through the fluidic device. The system was placed into an incubator or climate room at 37° C.

The cell growth and differentiation were checked under a microscope via the transparent parts of the fluidic device. After the desired level of cell differentiation was reached, several stimuli, e.g. immune cells, pathogen, control compounds, test compounds or the like, were added to the system and/or collected from the system. The formed cell culture perfusion system could be used for scientific and industrial needs, e.g. testing therapies to the constructed mammal tissue.

1-29. (canceled)

30. A fluidic device for in vitro complex living tissue reconstruction comprising:
at least one set of distinct compartments, such as a set of channels and/or microchannels, which set comprises at least a first, a second and a third compartment; and
a separating material separating the compartments comprised in the set of distinct compartments from one another, wherein:
the at least one set of distinct compartments defines at least one exchange region in which the compartments comprised in the set congregate; and
at least a part of the separating material comprised in the at least one exchange region is configured such that direct communication is allowed between each of the compartments comprised in the at least one set of distinct compartments with one another.

31. A fluidic device according to claim 30, wherein an interstitial space is enclosed by the outer surfaces of each of the compartments.

32. A fluidic device according to claim 30, wherein the separating material encloses an interstitial space.

33. A fluidic device according to claim 30, wherein at least a part of the separating material comprised in the at least one exchange region comprises a plurality of passages configured to allow mass transfer, such as cell migration, between each of the compartments comprised in the at least one set of distinct compartments with one another.

34. A fluidic device according to claim 30, wherein the separating material separating the compartments has a thickness from 0.5 μm or greater.

35. A fluidic device according to claim 30, wherein the minimal distance between each of the compartments in the at least one exchange region lies within the range of 10 to 250 μm.

36. A fluidic device according to claim 30, wherein the at least one set of distinct compartments further comprises a fourth compartment.

37. A fluidic device according to claim 30, wherein at least one of the compartments and/or the interstitial space further comprises at least one hollow membrane for culturing and/or co-culturing, evaluating, sampling and/or harvesting of tissue cells, circulatory system cells, neuronal cells, interstitial cells, products and/or metabolites from the fluidic device.

38. A fluidic device according to claim 37, wherein the hollow membrane is made of a permeable and/or semi-permeable material, and in particular the hollow membrane has a varied permeability per surface area of the hollow membrane.

39. A fluidic device according to claim 37, wherein the hollow membrane is at least partially made of a biodegradable material.

40. A perfusion system comprising the fluidic device according to claim 30.

41. A method for in vitro culturing and/or co-culturing cells, including complex living tissue reconstruction, comprising the following steps:
a) providing a perfusion system according to claim 40;
b) providing tissue cells, circulatory system cells and, optionally, neuronal cells;
c) allowing medium to flow through the fluidic device;
d) closing the inlet ports and outlet ports of the fluidic device to stop the flow of medium once the fluidic device is filled with medium;
e) seeding the tissue cells to the first compartment of the fluidic device;
f) seeding the circulatory system cells to the second compartment of the fluidic device;
g) optionally, seeding the neuronal cells to the third compartment of the fluidic device; and
h) open the inlet ports and outlet ports of the fluidic device to allow medium to flow through the fluidic device.

42. A hollow membrane for evaluating, sampling and/or harvesting of tissue cells, circulatory system cells, neuronal cells, interstitial cells, products and/or metabolites from the fluidic device according to claim 30, wherein the hollow membrane is made of a permeable and/or semi-permeable material, and in particular has a varied permeability per surface area of the hollow membrane.

43. A hollow membrane according to claim 42, wherein the hollow membrane is made of a biodegradable material.

44. A method for culturing, co-culturing, evaluating, sampling and/or harvesting tissue cells, circulatory system cells, neuronal cells, interstitial cells or products and/or metabolites, which method comprises applying said cells, products and/or metabolites to the hollow membrane of claim 42.
